(19) United States
(12) Patent Application Publication
(10) Pub. No.: US 2025/0283844 A1
(43) Pub. Date: Sep. 11, 2025

(54) GAS SENSOR, AND CONCENTRATION MEASUREMENT METHOD USING GAS SENSOR

(71) Applicant: NGK INSULATORS, LTD., Nagoya-shi (JP)

(72) Inventors: Yusuke WATANABE, Nagoya-shi (JP); Daichi ICHIKAWA, Nagoya-shi (JP)

(21) Appl. No.: 19/214,316

(22) Filed: May 21, 2025

Related U.S. Application Data
(63) Continuation of application No. PCT/JP2023/033742, filed on Sep. 15, 2023.

(30) Foreign Application Priority Data
Nov. 24, 2022 (JP) 2022-187223

(51) Int. Cl. G01N 27/27; G01N 27/407; G01N 27/41; G01N 27/417; G01N 33/00

(52) U.S. Cl. CPC G01N 27/27; G01N 27/4075; G01N 27/41; G01N 27/417; G01N 33/0037; G01N 33/004

(57) ABSTRACT

A sensor element includes first to fourth chambers communicating sequentially, an adjustment cell pumps oxygen out of a measurement gas introduced into the first chamber so that NOx, H₂O, and CO₂ are not decomposed, a first measurement cell pumps out oxygen from the second chamber so that all NOx is reduced, a second measurement cell pumps out oxygen from the third chamber so that all H₂O and CO₂ are reduced, a third measurement cell pumps oxygen into the fourth chamber to selectively oxidize H₂ generated by reduction, a concentration of NOx is identified from a current generated by pumping-out by the first measurement pump cell, a concentration of H₂O is identified from a current generated by pumping-in by the third measurement pump cell, and a concentration of CO₂ is identified based on the identified concentration of H₂O and a current generated by pumping-out by the second measurement pump cell.

### CROSS-REFERENCE TO RELATED APPLICATION

This application is a continuation application of PCT/JP2023/033742, filed on Sep. 15, 2023, which claims the benefit of priority of Japanese Application No. JP2022-187223, filed on Nov. 24, 2022, the entire contents of which are incorporated herein by reference.

### BACKGROUND

### Technical Field

The present invention relates to a multi-gas sensor capable of sensing a plurality of types of sensing target gas components and measuring concentrations thereof.

### Description of the Background Art

In measurement for managing the amount of an emitted exhaust gas from a vehicle, technology of measuring concentrations of water vapor (H₂O) and carbon dioxide (CO₂) has already been known (see Japanese Patent No. 5918177 and No. 6469464, for example). In each of gas sensors disclosed in Japanese Patent No. 5918177 and No. 6469464, a water vapor (H₂O) component and a carbon dioxide (CO₂) component can be measured in parallel.

A gas sensor (NOx sensor) capable of measuring NOx by performing different pump cell control from that performed in the gas sensors disclosed in Japanese Patent No. 5918177 and No. 6469464 while including a sensor element having a similar configuration to that of the gas sensors disclosed in Japanese Patent No. 5918177 and No. 6469464 has also already been known (see Japanese Patent No. 3798412for example).

In the gas sensor disclosed in Japanese Patent No. 5918177 having a three-chamber configuration, firstly, a main pump cell as a pump cell for a first internal space operates to pump out O₂ contained in a measurement gas introduced into the first internal space and to reduce all H₂O and CO₂ similarly contained in the measurement gas once to generate H₂ and CO. The measurement gas containing these H₂ and CO is introduced into a second internal space and further into a third internal space. A first measurement pump cell as a pump cell for the second internal space then pumps in O₂ to selectively oxidize H₂ to generate H₂O, and, further, a second measurement pump cell as a pump cell for the third internal space pumps in O₂ to oxidize CO to generate CO₂. Concentrations of H₂O and CO₂ in the measurement gas are respectively measured based on magnitudes of pump currents flowing through the first measurement pump cell and the second measurement pump cell when H₂ and CO are oxidized.

In the gas sensor, an applied voltage in the pump cell for the first internal space is required to be set to be high for reduction of H₂O and CO₂ in the first internal space. In addition, a temperature of a main inner pump electrode as an in-space pump electrode forming the main pump cell is required to be set to be high. Such a high applied voltage and maintaining the pump electrode at a high temperature, however, might cause a sensor element containing oxygen-ion conductive solid electrolyte ceramics as a major component to be cracked and blackened to reduce the solid electrolyte ceramics.

Furthermore, measurement of NOx is sometimes desired to be performed in parallel with measurement of H₂O and CO contained in an exhaust gas. In particular, in terms of ease of securement of an attachment space and cost efficiency, there is a need to simultaneously measure H₂O and CO as well as NOx using the same gas sensor.

### SUMMARY

The present invention relates to a multi-gas sensor capable of sensing a plurality of types of sensing target gas components and measuring concentrations thereof.

According to the present invention, a gas sensor capable of measuring concentrations of a plurality of sensing target gas components, includes: a sensor element having a structure formed of an oxygen-ion conductive solid electrolyte; and a controller controlling operation of the gas sensor, wherein the sensor element includes: a gas inlet through which a measurement gas is introduced; a first chamber, a second chamber, a third chamber, and a fourth chamber communicating sequentially from the gas inlet via different diffusion control parts; an adjustment pump cell including an inner electrode formed to face the first chamber, an out-of-space pump electrode provided at a location other than a location in the first chamber, the second chamber, the third chamber, and the fourth chamber, and a portion of the solid electrolyte present between the inner electrode and the out-of-space pump electrode; a first measurement pump cell including a first measurement electrode formed to face the second chamber, the out-of-space pump electrode, and a portion of the solid electrolyte present between the first measurement electrode and the out-of-space pump electrode; a second measurement pump cell including a second measurement electrode formed to face the third chamber, the out-of-space pump electrode, and a portion of the solid electrolyte present between the second measurement electrode and the out-of-space pump electrode; a third measurement pump cell including a third measurement electrode formed to face the fourth chamber, the out-of-space pump electrode, and a portion of the solid electrolyte present between the third measurement electrode and the out-of-space pump electrode; and a heater heating the sensor element, the inner electrode is a cermet electrode containing a Pt—Au alloy as a metal component, the Pt—Au alloy having an Au concentration of 0.5 wt % or more, the first measurement electrode is another cermet electrode containing a Pt—Rh alloy as a metal component, the adjustment pump cell pumps oxygen out of the measurement gas introduced through the gas inlet into the first chamber so that NOx, water vapor, and carbon dioxide contained in the measurement gas are not decomposed, the first measurement pump cell pumps out oxygen from the second chamber so that substantially all NOx contained in the measurement gas introduced from the first chamber into the second chamber is reduced, the second measurement pump cell pumps out oxygen from the third chamber so that substantially all water vapor and carbon dioxide contained in the measurement gas introduced from the second chamber into the third chamber are reduced, the third measurement pump cell pumps oxygen into the fourth chamber to selectively oxidize, in the fourth chamber, hydrogen having been generated by reduction of water vapor and contained in the measurement gas introduced from the third chamber into the fourth chamber, and the controller identifies: a concentration of NOx contained in the measurement gas based on a magnitude of a NOx detection current as an oxygen pump current flowing between the first measurement electrode and the out-of-space pump electrode when oxygen is pumped out from the second chamber by the first measurement pump cell; a concentration of water vapor contained in the measurement gas based on a value of a water vapor equivalent current as an oxygen pump current flowing between the third measurement electrode and the out-of-space pump electrode when hydrogen is oxidized with oxygen pumped into the forth chamber by the third measurement pump cell; and a concentration of carbon dioxide contained in the measurement gas based on the value of the water vapor equivalent current and a value of a total reducing current as an oxygen pump current flowing between the second measurement electrode and the out-of-space pump electrode when water vapor and carbon dioxide are reduced by the second measurement pump cell pumping out oxygen from the third chamber.

Another aspect of the present invention is a concentration measurement method of measuring concentrations of a plurality of sensing target gas components using a gas sensor, wherein the gas sensor includes a sensor element having an elongated planar structure formed of an oxygen-ion conductive solid electrolyte, the sensor element includes: a gas inlet through which a measurement gas is introduced; a first chamber, a second chamber, a third chamber, and a fourth chamber communicating sequentially from the gas inlet via different diffusion control parts; an adjustment pump cell including an inner electrode formed to face the first chamber, an out-of-space pump electrode provided at a location other than a location in the first chamber, the second chamber, the third chamber, and the fourth chamber, and a portion of the solid electrolyte present between the inner electrode and the out-of-space pump electrode; a first measurement pump cell including a first measurement electrode formed to face the second chamber, the out-of-space pump electrode, and a portion of the solid electrolyte present between the first measurement electrode and the out-of-space pump electrode; a second measurement pump cell including a second measurement electrode formed to face the third chamber, the out-of-space pump electrode, and a portion of the solid electrolyte present between the second measurement electrode and the out-of-space pump electrode; a third measurement pump cell including a third measurement electrode formed to face the fourth chamber, the out-of-space pump electrode, and a portion of the solid electrolyte present between the third measurement electrode and the out-of-space pump electrode; and a heater heating the sensor element, the inner electrode is a cermet electrode containing a Pt—Au alloy as a metal component, the Pt—Au alloy having an Au concentration of 0.5 wt % or more, the first measurement electrode is another cermet electrode containing a Pt—Rh alloy as a metal component, and the concentration measurement method using the gas sensor includes: a) pumping, using the adjustment pump cell, oxygen out of the measurement gas introduced through the gas inlet into the first chamber so that NOx, water vapor, and carbon dioxide contained in the measurement gas are not decomposed; b) pumping out, using the first measurement pump cell, oxygen from the second chamber so that substantially all NOx contained in the measurement gas introduced from the first chamber into the second chamber is reduced; c) pumping out, using the second measurement pump cell, oxygen from the third chamber so that substantially all water vapor and carbon dioxide contained in the measurement gas introduced from the second chamber into the third chamber are reduced; d) pumping, using the third measurement pump cell, oxygen into the fourth chamber to selectively oxidize, in the fourth chamber, hydrogen having been generated by reduction of water vapor and contained in the measurement gas introduced from the third chamber into the fourth chamber; e) identifying a concentration of NOx contained in the measurement gas based on a magnitude of a NOx detection current as an oxygen pump current flowing between the first measurement electrode and the out-of-space pump electrode when NOx is reduced by pumping out oxygen from the second chamber using the first measurement pump cell; f) identifying a concentration of water vapor contained in the measurement gas based on a value of a water vapor equivalent current as an oxygen pump current flowing between the third measurement electrode and the out-of-space pump electrode when hydrogen is oxidized with oxygen pumped into the fourth chamber using the third measurement pump cell; and g) identifying a concentration of carbon dioxide contained in the measurement gas based on the value of the water vapor equivalent current and a value of a total reducing current as an oxygen pump current flowing between the second measurement electrode and the out-of-space pump electrode when water vapor and carbon dioxide are reduced by pumping out oxygen from the third chamber using the second measurement pump cell.

According to the present invention, a multi-gas sensor having more long-term reliability than before and capable of simultaneously measuring many types of gases is implemented.

It is therefore an object of the present invention to provide a multi-gas sensor with more long-term reliability than before capable of simultaneously measuring a water vapor (H₂O) component and a carbon dioxide (CO₂) component as well as NOx, suppressing cracking and blackening of a sensor element, and further being less likely to be subjected to a change in sensitivity during a long-term use.

### BRIEF DESCRIPTION OF DRAWINGS

FIG. 1 is a diagram schematically showing one example of a configuration of a gas sensor 100.

FIG. 2 is a block diagram showing functional components implemented by a controller 110.

FIG. 3 is a schematic diagram illustrating entry and exit of gases into and from four chambers comprised in a sensor element 101 of the gas sensor 100.

FIG. 4 is a graph showing a relationship between a target value of electromotive force V0 in a first chamber sensor cell 80 and an oxygen pump current Ip0 flowing through an adjustment pump cell 21 when three different types of model gases are caused to flow.

FIG. 5 is a diagram showing dependence of an oxygen pump current Ip2 on a concentration of a sensing target gas component.

FIG. 6 is a diagram showing dependence of an oxygen pump current Ip3 on a concentration of a sensing target gas component.

FIG. 7 is a diagram showing an example of H₂O characteristics data.

### DESCRIPTION OF THE PREFERRED EMBODIMENTS

### Configuration of Gas Sensor

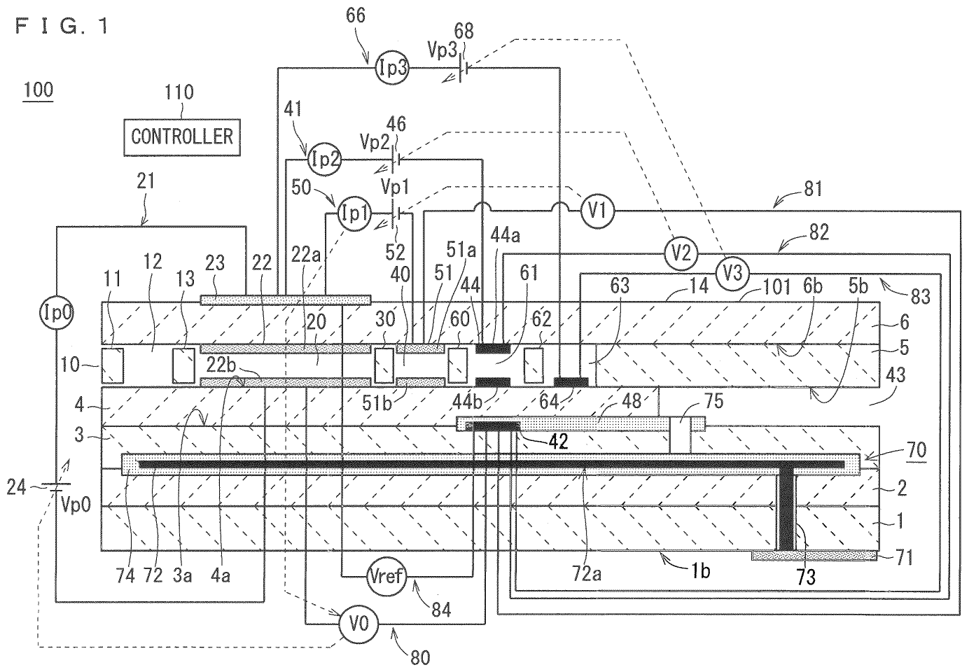

FIG. 1 is a diagram schematically showing one example of a configuration of a gas sensor 100 according to the present embodiment. The gas sensor 100 is a multi-gas sensor sensing a plurality of types of gas components and measuring concentrations thereof using a sensor element 101. Assume that at least water vapor (H₂O), carbon dioxide (CO₂), and nitrogen oxide (NOx) are main sensing target gas components of the gas sensor 100 in the present embodiment. The gas sensor 100 is attached to an exhaust path of an internal combustion engine, such as an engine of a vehicle, and is used with an exhaust gas flowing along the exhaust path as a measurement gas, for example. FIG. 1 includes a vertical cross-sectional view taken along a longitudinal direction of the sensor element 101.

The sensor element 101 includes an elongated planar structure (base part) 14 formed of an oxygen-ion conductive solid electrolyte, a first diffusion control part 11 doubling a gas inlet 10 which is formed in one end portion (a left end portion in the figure) of the structure 14 and through which the measurement gas is introduced, and a buffer space 12, a first chamber 20, a second chamber 40, a third chamber 61, and a fourth chamber 63 formed in the structure 14 and communicating sequentially from the gas inlet 10 (first diffusion control part 11). The buffer space 12 communicates with the gas inlet 10 (first diffusion control part 11). The first chamber 20 communicates with the buffer space 12 via a second diffusion control part 13. The second chamber 40 communicates with the first chamber 20 via a third diffusion control part 30. The third chamber 61 communicates with the second chamber 40 via a fourth diffusion control part 60. The fourth chamber 63 communicates with the third chamber 61 via a fifth diffusion control part 62.

The structure 14 is formed by laminating a plurality of substrates of ceramics, for example. Specifically, the structure 14 has a configuration in which six layers including a first substrate 1, a second substrate 2, a third substrate 3, a first solid electrolyte layer 4, a spacer layer 5, and a second solid electrolyte layer 6 are sequentially laminated from the bottom. Each layer is formed of an oxygen-ion conductive solid electrolyte, such as zirconia (ZrO₂).

The first diffusion control part 11 doubling as the gas inlet 10, the buffer space 12, the second diffusion control part 13, the first chamber 20, the third diffusion control part 30, the second chamber 40, the fourth diffusion control part 60, the third chamber 61, the fifth diffusion control part 62, and the fourth chamber 63 are formed in this order between a lower surface 6b of the second solid electrolyte layer 6 and an upper surface 4a of the first solid electrolyte layer 4 on a side of the one end portion of the structure 14. A part extending from the gas inlet 10 to the fourth chamber 63 is also referred to as a gas distribution part.

The buffer space 12, the first chamber 20, the second chamber 40, the third chamber 61, and the fourth chamber 63 are formed to penetrate the spacer layer 5 in a thickness direction. The lower surface 6b of the second solid electrolyte layer 6 is exposed in upper portions in the figure of these chambers and the like, and the upper surface 4a of the first solid electrolyte layer 4 is exposed in lower portions in the figure of these chambers and the like. Side portions of these chambers and the like are each defined by the spacer layer 5 or any of the diffusion control parts. The first chamber 20, the second chamber 40, the third chamber 61, and the fourth chamber 63 each have a length (size in the longitudinal direction of the element) of 0.3 mm to 1.0 mm, for example, a width (size in a transverse direction of the element) of 0.5 mm to 30 mm, for example, and a height (size in a thickness direction of the element) of 50 μm to 200 μm, for example. These chambers, however, are not required to have the same size and may have different sizes.

The gas inlet 10 may similarly be formed to penetrate the spacer layer 5 in the thickness direction separately from the first diffusion control part 11. In this case, the first diffusion control part 11 is to be formed inside and adjacent to the gas inlet 10.

The first diffusion control part 11, the second diffusion control part 13, the third diffusion control part 30, the fourth diffusion control part 60, and the fifth diffusion control part 62 each include two horizontally long slits. That is to say, they each have openings elongated in a direction perpendicular to the page of the figure in an upper portion and a lower portion in the figure thereof. The slits each have a length (size in the longitudinal direction of the element) of 0.2 mm to 1.0 mm, for example, a width of an opening (size in the transverse direction of the element) of 0.5 mm to 30 mm, for example, and a height of the opening (size in the thickness direction of the element) of 5 μm to 30 μm, for example.

The sensor element 101 includes a reference gas introduction space 43 in the other end portion (a right end portion in the figure) opposite the one end portion in which the gas inlet 10 is provided. The reference gas introduction space 43 is formed between an upper surface 3a of the third substrate 3 and a lower surface 5b of the spacer layer 5. A side portion of the reference gas introduction space 43 is defined by a side surface of the first solid electrolyte layer 4. Oxygen (O₂) and air are introduced into the reference gas introduction space 43 as reference gases, for example.

The gas inlet 10 (first diffusion control part 11) is a part opening to an external space, and the measurement gas is taken from the external space into the sensor element 101 through the gas inlet 10.

The first diffusion control part 11 is a part providing predetermined diffusion resistance to the taken measurement gas.

The buffer space 12 is provided to cancel concentration fluctuations of the measurement gas caused by pressure fluctuations of the measurement gas in the external space. Pulsation of exhaust pressure of the exhaust gas of the vehicle is taken as an example of such pressure fluctuations of the measurement gas, for example.

The second diffusion control part 13 is a part providing predetermined diffusion resistance to the measurement gas introduced from the buffer space 12 into the first chamber 20.

The first chamber 20 is provided as a space to pump oxygen out of the measurement gas introduced through the second diffusion control part 13. Pumping-out of oxygen is implemented by operation of an adjustment pump cell 21.

The adjustment pump cell 21 is an electrochemical pump cell including an inner pump electrode (adjustment electrode) 22, an outer pump electrode (out-of-space pump electrode) 23, and a solid electrolyte present in a portion of the structure 14 sandwiched between these electrodes.

In the adjustment pump cell 21, a voltage Vp0 is applied across the inner pump electrode 22 and the outer pump electrode 23 from a variable power supply 24 disposed outside the sensor element 101 to generate an oxygen pump current (oxygen ion current) Ip0. Oxygen in the first chamber 20 can thereby be pumped out to the external space. Assume that a direction of the oxygen pump current Ip0 when oxygen is pumped out from the first chamber 20 is a positive direction of the oxygen pump current Ip0 in the present embodiment.

The inner pump electrode 22 is provided on substantially the entire portions of the lower surface 6b of the second solid electrolyte layer 6 and the upper surface 4a of the first solid electrolyte layer 4 defining the first chamber 20 respectively as a ceiling electrode portion 22a and a bottom electrode portion 22b. The ceiling electrode portion 22a and the bottom electrode portion 22b are connected by an unillustrated conducting portion.

The inner pump electrode 22 is provided, with an alloy (a Pt—Au alloy) of platinum (Pt) and gold (Au) inert to NOx as a metal component, as a porous cermet electrode containing the Pt—Au alloy and zirconia and being rectangular in plan view, for example. The Pt—Au alloy preferably contains Au at a concentration of 0.5 wt % or more to surely pump out only oxygen contained in the measurement gas without reducing NOx, H₂O, and CO₂.

The outer pump electrode 23 is provided, with platinum or the Pt—Au alloy as a metal component, as a porous cermet electrode containing platinum or the Pt—Au alloy and zirconia and being rectangular in plan view, for example.

In the sensor element 101, the inner pump electrode 22, a reference electrode 42, and a solid electrolyte present in a portion of the structure 14 sandwiched between these electrodes constitute a first chamber sensor cell 80. The first chamber sensor cell 80 is an electrochemical sensor cell to grasp oxygen partial pressure in an atmosphere in the first chamber 20.

The reference electrode 42 is an electrode formed between the first solid electrolyte layer 4 and the third substrate 3 and is provided as a porous cermet electrode containing platinum and zirconia and being rectangular in plan view, for example.

A reference gas introduction layer 48 formed of porous alumina and leading to the reference gas introduction space 43 is provided around the reference electrode 42. A reference gas in the reference gas introduction space 43 is introduced into a surface of the reference electrode 42 via the reference gas introduction layer 48. That is to say, the reference electrode 42 is always in contact with the reference gas.

In the first chamber sensor cell 80, electromotive force (Nernst electromotive force) V0 is generated between the inner pump electrode 22 and the reference electrode 42. The electromotive force V0 has a value in accordance with a difference between an oxygen concentration (oxygen partial pressure) in the first chamber 20 and an oxygen concentration (oxygen partial pressure) of the reference gas. The oxygen concentration (oxygen partial pressure) of the reference gas is basically constant, so that the electromotive force V0 has a value in accordance with the oxygen concentration (oxygen partial pressure) in the first chamber 20.

The third diffusion control part 30 is a part providing predetermined diffusion resistance to the measurement gas introduced from the first chamber 20 into the second chamber 40.

The second chamber 40 is provided as a space to reduce (decompose) NOx contained as a sensing target gas component in the measurement gas introduced through the third diffusion control part 30 and pump out oxygen thus generated, so that the measurement gas contains H₂O and CO₂ but does not substantially contain NOx. Pumping-out of oxygen is implemented by operation of a first measurement pump cell 50.

The first measurement pump cell 50 is an electrochemical pump cell including a first measurement electrode 51, the outer pump electrode 23, and a solid electrolyte present in a portion of the structure 14 sandwiched between these electrodes.

In the first measurement pump cell 50, a voltage Vp1 is applied across the first measurement electrode 51 and the outer pump electrode 23 from a variable power supply 52 disposed outside the sensor element 101 to generate an oxygen pump current (oxygen ion current) Ip1. Oxygen in the second chamber 40 can thereby be pumped out to the external space. Assume that a direction of the oxygen pump current Ip1 when oxygen is pumped out from the second chamber 40 is a positive direction of the oxygen pump current Ip1 in the present embodiment.

The first measurement electrode 51 is provided on substantially the entire portions of the lower surface 6b of the second solid electrolyte layer 6 and the upper surface 4a of the first solid electrolyte layer 4 defining the second chamber 40 respectively as a ceiling electrode portion 51a and a bottom electrode portion 51b. The ceiling electrode portion 51a and the bottom electrode portion 51b are connected by an unillustrated conducting portion.

The first measurement electrode 51 is provided, with an alloy (a Pt—Rh alloy) of platinum and rhodium (Rh) as a metal component, as a porous cermet electrode containing the Pt—Rh alloy and zirconia and being rectangular in plan view, for example. The Pt—Rh alloy preferably has an Rh concentration of 30 wt % or more.

In the sensor element 101, the first measurement electrode 51, the reference electrode 42, and a solid electrolyte present in a portion of the structure 14 sandwiched between these electrodes constitute a second chamber sensor cell 81. The second chamber sensor cell 81 is an electrochemical sensor cell to grasp oxygen partial pressure in an atmosphere in the second chamber 40.

In the second chamber sensor cell 81, electromotive force (Nernst electromotive force) V1 is generated between the first measurement electrode 51 and the reference electrode 42. The electromotive force V1 has a value in accordance with a difference between an oxygen concentration (oxygen partial pressure) in the second chamber 40 and the oxygen concentration (oxygen partial pressure) of the reference gas. Since the oxygen concentration (oxygen partial pressure) of the reference gas is basically constant, the electromotive force V1 has a value in accordance with the oxygen concentration (oxygen partial pressure) in the second chamber 40.

The fourth diffusion control part 60 is a part providing predetermined diffusion resistance to the measurement gas introduced from the second chamber 40 into the third chamber 61, containing H₂O and CO₂, and not substantially containing NOx and oxygen.

The third chamber 61 is provided as a space to reduce (decompose) H₂O and CO₂ contained as sensing target gas components in the measurement gas introduced through the fourth diffusion control part 60 to generate hydrogen (H₂) and carbon monoxide (CO), so that the measurement gas does not substantially contain NOx and oxygen as well as H₂O and CO₂. Reduction (decomposition) of H₂O and CO₂ is implemented by operation of a second measurement pump cell 41.

The second measurement pump cell 41 is an electrochemical pump cell including a second measurement electrode 44, the outer pump electrode 23, and a solid electrolyte present in a portion of the structure 14 sandwiched between these electrodes.

In the second measurement pump cell 41, a voltage Vp2 is applied across the second measurement electrode 44 and the outer pump electrode 23 from a variable power supply 46 disposed outside the sensor element 101 to generate an oxygen pump current (oxygen ion current) Ip2. Oxygen generated in the third chamber 61 by reduction of H₂O and CO₂ can thereby be pumped out to the external space. Assume that a direction of the oxygen pump current Ip2 when oxygen is pumped out from the third chamber 61 is a positive direction of the oxygen pump current Ip2 in the present embodiment.

The second measurement electrode 44 is provided on substantially the entire portions of the lower surface 6b of the second solid electrolyte layer 6 and the upper surface 4a of the first solid electrolyte layer 4 defining the third chamber 61 respectively as a ceiling electrode portion 44a and a bottom electrode portion 44b. The ceiling electrode portion 44a and the bottom electrode portion 44b are connected by an unillustrated conducting portion.

The second measurement electrode 44 is provided as a porous cermet electrode containing Pt as a metal component and being rectangular in plan view.

In the sensor element 101, the second measurement electrode 44, the reference electrode 42, and a solid electrolyte present in a portion of the structure 14 sandwiched between these electrodes constitute a third chamber sensor cell 82. The third chamber sensor cell 82 is an electrochemical sensor cell to grasp oxygen partial pressure in an atmosphere in the third chamber 61.

In the third chamber sensor cell 82, electromotive force (Nernst electromotive force) V2 is generated between the second measurement electrode 44 and the reference electrode 42. The electromotive force V2 has a value in accordance with a difference between an oxygen concentration (oxygen partial pressure) in the third chamber 61 and the oxygen concentration (oxygen partial pressure) of the reference gas. Since the oxygen concentration (oxygen partial pressure) of the reference gas is basically constant, the electromotive force V2 has a value in accordance with the oxygen concentration (oxygen partial pressure) in the third chamber 61.

The fifth diffusion control part 62 is a part providing predetermined diffusion resistance to the measurement gas introduced from the third chamber 61 into the fourth chamber 63 and containing H₂ and CO while not substantially containing H₂O, CO₂, NOx, and oxygen.

The fourth chamber 63 is provided as a space to selectively oxidize all of H₂ from among H₂ and CO contained in the measurement gas introduced through the fifth diffusion control part 62 to generate H₂O again. Generation of H₂O by oxidation of H₂ is implemented by operation of a third measurement pump cell 66.

The third measurement pump cell 66 is an electrochemical pump cell including a third measurement electrode 64, the outer pump electrode 23, and a solid electrolyte present in a portion of the structure 14 sandwiched between these electrodes.

In the third measurement pump cell 66, a voltage Vp3 is applied across the third measurement electrode 64 and the outer pump electrode 23 from a variable power supply 68 disposed outside the sensor element 101 to generate an oxygen pump current (oxygen ion current) Ip3. Oxygen can thereby be pumped into the fourth chamber 63 from the external space. Assume that a direction of the oxygen pump current Ip3 when oxygen is pumped out from the fourth chamber 63 is a positive direction of the oxygen pump current Ip3 in the present embodiment.

The third measurement electrode 64 is provided on substantially the entire portion of the upper surface 4a of the first solid electrolyte layer 4 defining the fourth chamber 63.

The third measurement electrode 64 is provided, with the Pt—Au alloy as a metal component, as a porous cermet electrode containing the Pt—Au alloy and zirconia and being rectangular in plan view. The Pt—Au alloy preferably has an Au concentration of 1 wt % or more and 50 wt % or less and more preferably has an Au concentration of 10 wt % or more and 30 wt % or less. In this case, a selective H₂ oxidation property, that is, a property that, when H₂ and CO coexist in the fourth chamber 63, only H₂ is selectively oxidized with oxygen pumped in by the third measurement pump cell 66 and CO is not oxidized, of the third measurement electrode 64 is more suitably developed.

In the sensor element 101, the third measurement electrode 64, the reference electrode 42, and a solid electrolyte present in a portion of the structure 14 sandwiched between these electrodes constitute a fourth chamber sensor cell 83. The fourth chamber sensor cell 83 is an electrochemical sensor cell to grasp oxygen partial pressure in an atmosphere in the fourth chamber 63.

In the fourth chamber sensor cell 83, electromotive force (Nernst electromotive force) V3 is generated between the third measurement electrode 64 and the reference electrode 42. The electromotive force V3 has a value in accordance with a difference between an oxygen concentration (oxygen partial pressure) in the fourth chamber 63 and the oxygen concentration (oxygen partial pressure) of the reference gas. Since the oxygen concentration (oxygen partial pressure) of the reference gas is basically constant, the electromotive force V3 has a value in accordance with the oxygen concentration (oxygen partial pressure) in the fourth chamber 63.

The sensor element 101 further includes an electrochemical sensor cell 84 including the outer pump electrode 23, the reference electrode 42, and a solid electrolyte present in a portion of the structure 14 sandwiched between these electrodes. Electromotive force Vref generated between the outer pump electrode 23 and the reference electrode 42 of the sensor cell 84 has a value in accordance with oxygen partial pressure of the measurement gas present outside the sensor element 101.

In addition to the foregoing, the sensor element 101 includes a heater part 70 playing a role in temperature adjustment of heating the sensor element 101 and maintaining the temperature thereof to enhance oxygen ion conductivity of the solid electrolyte forming the structure 14.

The heater part 70 mainly includes a heater electrode 71, a heater element 72, a heater lead 72a, a through hole 73, a heater insulating layer 74, and a heater resistance detection lead, which is not illustrated in FIG. 1. The heater element 72 is hereinafter also simply referred to as a heater 72.

The heater 72 is provided to be sandwiched between the second substrate 2 and the third substrate 3 from below and above and generates heat by being powered from outside through the heater electrode 71 provided on a lower surface 1b of the first substrate 1, the through hole 73, and the heater lead 72a. The heater 72 is buried over the entire region of a range from the buffer space 12 to the fourth chamber 63 and can heat the sensor element 101 to a predetermined temperature and, further, maintain the temperature.

The heater 72 is provided so that a temperature is highest near the first chamber 20 (near the inner pump electrode 22) and decreases with increasing distance from the first chamber 20 in the longitudinal direction of the element during heating. In the present embodiment, a temperature in a range from the one end portion of the sensor element 101 in which the gas inlet 10 is disposed to the fourth chamber 63 when the gas sensor 100 is in use (when the sensor element 101 is driven) is referred to as an element driving temperature. The heater 72 performs heating so that the element driving temperature is in a range of 750° C. to 950° C.

The heater insulating layer 74 of alumina and the like is formed above and below the heater 72 to electrically insulate the heater 72 from the second substrate 2 and the third substrate 3. The heater part 70 also includes a pressure dissipation hole 75. The pressure dissipation hole 75 is a part provided to penetrate the third substrate 3 and communicate with the reference gas introduction space 43 and is provided to mitigate a rise in internal pressure associated with a rise in temperature in the heater insulating layer 74.

The gas sensor 100 further includes a controller 110 controlling operation of the sensor element 101 and performing processing to identify concentrations of the sensing target gas components based on currents flowing through the sensor element 101.

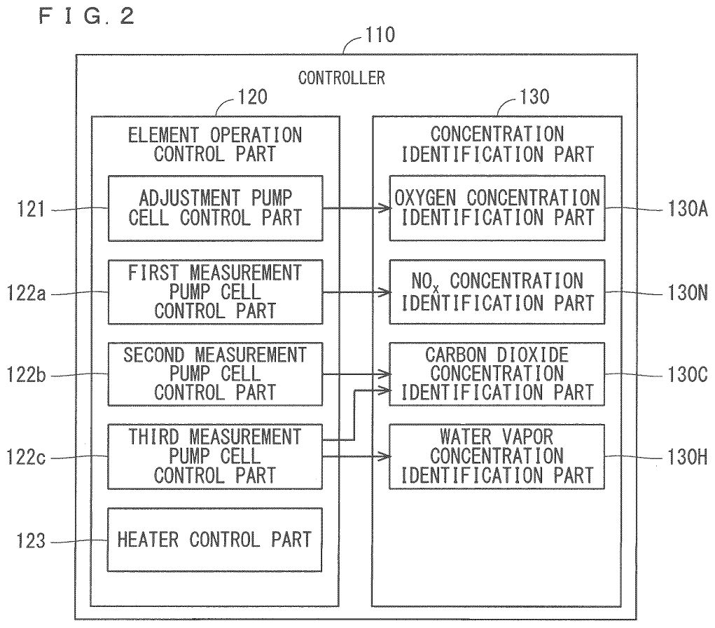

FIG. 2 is a block diagram showing functional components implemented by the controller 110. The controller 110 is configured by one or more electronic circuits including one or more central processing units (CPUs), a storage device, and the like, for example. Each of the electronic circuits is a software functional part implementing a predetermined functional component by a CPU executing a predetermined program stored in the storage device, for example. The controller 110 may naturally be configured by an integrated circuit, such as a field-programmable gate array (FPGA), on which a plurality of electronic circuits are connected in accordance with their functions and the like.

When the gas sensor 100 is attached to the exhaust path of the engine of the vehicle and is used with the exhaust gas flowing along the exhaust path as the measurement gas, some or all of functions of the controller 110 may be implemented by an electronic control unit (ECU) of the vehicle.

The controller 110 includes, as functional components implemented by the CPU executing a predetermined program, an element operation control part 120 controlling operation of each part of the sensor element 101 described above and a concentration identification part 130 performing processing to identify the concentrations of the sensing target gas components contained in the measurement gas.

The element operation control part 120 mainly includes an adjustment pump cell control part 121 controlling operation of the adjustment pump cell 21, a first measurement pump cell control part 122a controlling operation of the first measurement pump cell 50, a second measurement pump cell control part 122b controlling operation of the second measurement pump cell 41, a third measurement pump cell control part 122c controlling operation of the third measurement pump cell 66, and a heater control part 123 controlling heating operation performed by the heater 72.

On the other hand, the concentration identification part 130 mainly includes a NOx concentration identification part 130N, a water vapor concentration identification part 130H, and a carbon dioxide concentration identification part 130C respectively identifying concentrations of NOx, H₂O, and CO₂ as the main sensing target gas components of the gas sensor 100.

The NOx concentration identification part 130N identifies the concentration of NOx contained in the measurement gas based on a value of the oxygen pump current Ip1 flowing through the first measurement pump cell 50 acquired by the first measurement pump cell control part 122a.

The water vapor concentration identification part 130H identifies the concentration of H₂O contained in the measurement gas based on a value of the oxygen pump current Ip3 flowing through the third measurement pump cell 66 acquired by the third measurement pump cell control part 122c.

The carbon dioxide concentration identification part 130C identifies the concentration of CO₂ contained in the measurement gas based on the concentration of H₂O identified by the water vapor concentration identification part 130H (the value of the oxygen pump current Ip3 based on which the concentration of H₂O is identified) and a value of the oxygen pump current Ip2 flowing through the second measurement pump cell 41 acquired by the second measurement pump cell control part 122b.

The concentration identification part 130 further includes an oxygen concentration identification part 130A identifying a concentration of oxygen contained in the measurement gas. The oxygen concentration identification part 130A identifies the concentration of oxygen contained in the measurement gas based on a value of the oxygen pump current Ip0 flowing through the adjustment pump cell 21 acquired by the adjustment pump cell control part 121. That is to say, the gas sensor 100 according to the present embodiment senses, in addition to NOx, H₂O, and CO₂ as the main sensing target gas components, oxygen as an appendant sensing target gas component.

### Multi-Gas Sensing and Concentration Identification

A method of sensing a plurality of types of gases (multi-gas sensing) and identifying concentrations of the sensed gases implemented by the gas sensor 100 having a configuration as described above will be described next. Assume hereinafter that the measurement gas is an exhaust gas containing oxygen, NOx, H₂O, and CO₂.

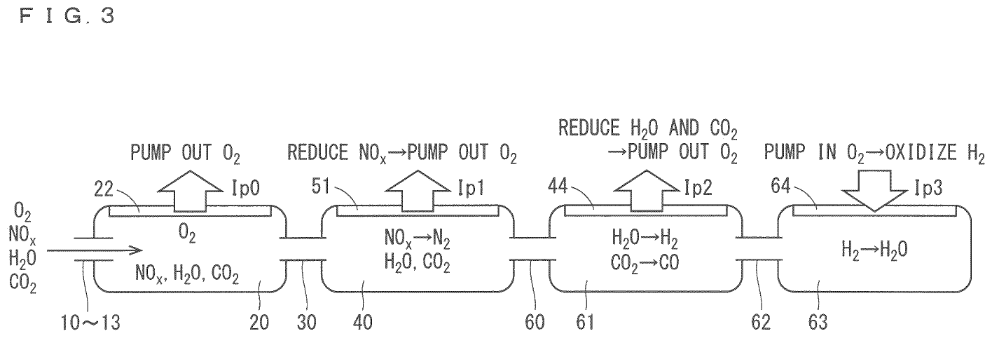

FIG. 3 is a schematic diagram illustrating entry and exit of gases into and from the four chambers (internal spaces) comprised in the sensor element 101 of the gas sensor 100.

First, in the sensor element 101 of the gas sensor 100 according to the present embodiment, the measurement gas is introduced through the gas inlet 10 (first diffusion control part 11), the buffer space 12, and the second diffusion control part 13 into the first chamber 20 as described above. In the first chamber 20, oxygen is pumped out of the introduced measurement gas by operation of the adjustment pump cell 21.

Pumping-out of oxygen is performed in the way that the adjustment pump cell control part 121 of the controller 110 sets a target value (control voltage) of the electromotive force V0 in the first chamber sensor cell 80 to a value in a range of 300 mV to 500 mV (preferably 350 mV) and feedback-controls the voltage Vp0 applied from the variable power supply 24 to the adjustment pump cell 21 in accordance with a difference between an actual value and the target value of the electromotive force V0 so that the electromotive force V0 is maintained at the target value. A value of the electromotive force V0 significantly deviates from the target value when the measurement gas containing a large amount of oxygen reaches the first chamber 20, for example, and thus the adjustment pump cell control part 121 controls the pump voltage Vp0 applied from the variable power supply 24 to the adjustment pump cell 21 so that the deviation is reduced.

The adjustment pump cell 21 pumps out oxygen from the first chamber 20 in such a manner, so that oxygen partial pressure (concentration) in the first chamber 20 is maintained at a sufficiently low value to the extent that H₂O and CO₂ contained in the measurement gas are not reduced. For example, the oxygen concentration in the first chamber 20 is approximately 0.1 ppm to 0.00001 ppm by operation of the adjustment pump cell 21.

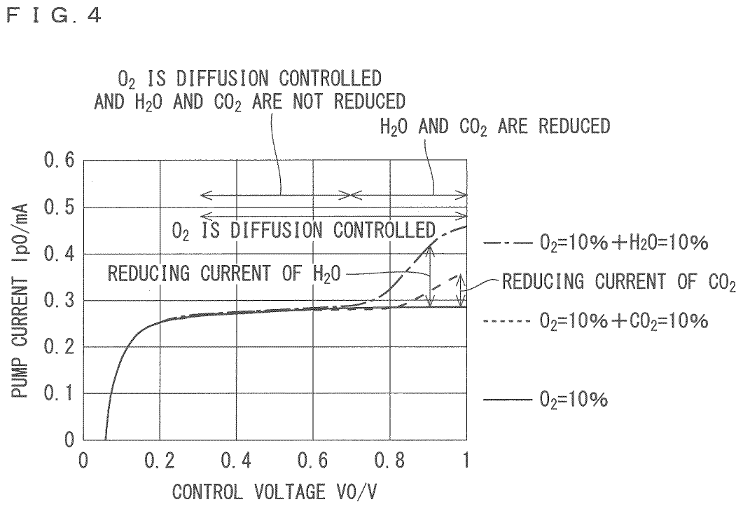

FIG. 4 is a diagram for describing a reason why oxygen is pumped out to the extent that H₂O and CO₂ are not reduced by setting the target value of the electromotive force V0 to the value in the range of 300 mV to 500 mV. Specifically, FIG. 4 is a graph showing a relationship between the target value (control voltage) of the electromotive force V0 in the first chamber sensor cell 80 and the oxygen pump current Ip0 flowing through the adjustment pump cell 21 when three different types of model gases are caused to flow. Specifically, the three types of model gases include a first gas containing oxygen of 10%, a second gas containing oxygen of 10% and CO₂ of 10%, and a third gas containing oxygen of 10% and H₂O of 10%. The balance of each of the gases is nitrogen (N₂). The element driving temperature is 800° C. or more, and a temperature of each of the model gases is 150° C.

It can be seen from FIG. 4 that, in a case of the first gas, the oxygen pump current Ip0 is substantially constant when the control voltage is in a range of 0.3 V or more, while, in a case of the second gas and the third gas, a profile is substantially the same as that for the first gas when the control voltage is in a range of 0.7 V or less but the oxygen pump current Ip0 increases again when the control voltage exceeds 0.7 V. The increase results from superimposition of reducing currents of H₂O or CO₂ flowing when H₂O or CO₂ contained in the measurement gas is reduced (decomposed) to generate oxygen.

As such, the target value of the electromotive force V0 is set to the value in the range of 300 mV to 500 mV in the present embodiment.

Similarly to H₂O and CO₂, NOx is not reduced with pumping-out of oxygen by the adjustment pump cell 21. This, however, is not due to setting of the target value of the electromotive force V0 but because the inner pump electrode 22 contains Au inert to NOx as described above.

As described above, in the gas sensor 100 according to the present embodiment, only pumping-out of oxygen in a manner that H₂O and CO₂ are not reduced is performed in the first chamber 20 to be at a highest temperature in the sensor element 101 during operation, and reduction of H₂O and CO₂ is not performed, in contrast to a gas sensor in conventional technology. Furthermore, NOx is also not reduced.

The target value of the electromotive force V0 in the first chamber sensor cell 80 set for pumping-out of oxygen is 300 mV to 500 mV, which is sufficiently smaller than a target value of 1000 mV to 1500 mV set when H₂O and CO₂ are reduced. An increase in pump voltage Vp0 is thus suppressed compared with a voltage applied to a corresponding pump cell in the gas sensor in conventional technology accompanied by reduction of H₂O and CO₂. Cracking and blackening attributable to application of a high voltage with the inner pump electrode 22 being maintained at a high temperature are thus suitably suppressed in the gas sensor 100 according to the present embodiment.

Since the inner pump electrode 22 contains Au inert to NOx, NOx is not reduced with pumping-out of oxygen by the adjustment pump cell 21 even when the measurement gas contains NOx.

The measurement gas of which only oxygen has been pumped out in the first chamber 20 to the extent that NOx, H₂O, and CO₂ are not reduced is introduced into the second chamber 40. NOx contained in the measurement gas is reduced in the second chamber 40. The first measurement electrode 51 disposed in the second chamber 40 and forming the first measurement pump cell 50 contains the Pt—Rh alloy as the metal component and does not contain Au inert to NOx, so that reduction of NOx progresses in the first measurement electrode 51. That is to say, oxygen is further pumped out of the measurement gas of which oxygen has been pumped out in the first chamber 20 and which is then introduced into the second chamber 40 by operation of the first measurement pump cell 50, so that a reduction (decomposition) reaction (e.g., 2NO→2N₂+O₂) of NOx contained in the measurement gas progresses, and substantially all NO is decomposed into nitrogen and oxygen.

Reduction (decomposition) of NOx and pumping-out of oxygen thus generated are performed in the way that the first measurement pump cell control part 122a of the controller 110 sets a target value (control voltage) of the electromotive force V1 in the second chamber sensor cell 81 to a value in a range of 350 mV to 700 mV (preferably 400 mV) and feedback-controls the voltage Vp1 applied from the variable power supply 52 to the first measurement pump cell 50 in accordance with a difference between an actual value and the target value of the electromotive force V1 so that the electromotive force V1 is maintained at the target value.

The first measurement pump cell 50 operates in this manner, so that the oxygen partial pressure in the second chamber 40 is maintained at a similar value to or a slightly lower value than that in the first chamber 20. It is approximately 10⁻⁸ atm when an equation V2=400 mV holds true, for example. The measurement gas thus no longer substantially contains NOx and oxygen while containing H₂O and CO₂ (and further N₂).

In the gas sensor 100 according to the present embodiment, the concentration of NOx in the measurement gas is identified based on the oxygen pump current Ip1 flowing through the first measurement pump cell 50 during pumping-out of oxygen including reduction of NOx.

The oxygen pump current Ip1 (hereinafter also referred to as a NOx detection current Ip1) in the first measurement pump cell 50 flows with pumping-out of oxygen generated by decomposition of NOx contained in the measurement gas. A magnitude of the NOx detection current Ip1 is thus substantially proportional to the concentration of NOx contained in the measurement gas introduced through the gas inlet 10. That is to say, there is a linear relationship between the NOx detection current Ip1 and the concentration of NOx in the measurement gas. Data (Ip1-NOx data) indicating the linear relationship is identified in advance using model gases having known NOx concentrations and is stored in the controller 110.

During actual measurement using the gas sensor 100, the NOx concentration identification part 130N acquires a value of the NOx detection current Ip1 from the first measurement pump cell control part 122a. A value of the concentration of NOx corresponding to the acquired NOx detection current Ip1 is identified with reference to the Ip1-NOx data. The concentration of NOx in the measurement gas is thereby identified.

The measurement gas in which NOx has been reduced in the second chamber 40 to the extent that H₂O and CO₂ are not reduced is introduced into the third chamber 61. H₂O and CO₂ contained in the measurement gas are reduced in the third chamber 61. That is to say, oxygen is further pumped out of the measurement gas of which oxygen has been pumped out in the first chamber 20 and in which NOx has been reduced in the second chamber 40 by operation of the second measurement pump cell 41, so that a reduction (decomposition) reaction (2H₂O→2H₂+O₂ and 2CO₂→2CO+O₂) of H₂O and CO₂ contained in the measurement gas progresses, and substantially all H₂O and CO₂ are decomposed into hydrogen (H₂), carbon monoxide (CO), and oxygen.

Reduction (decomposition) of H₂O and CO₂ and pumping-out of oxygen as generated are performed in the way that the second measurement pump cell control part 122b of the controller 110 sets a target value (control voltage) of the electromotive force V2 in the third chamber sensor cell 82 to a value in a range of 1000 mV to 1500 mV (preferably 1000 mV) and feedback-controls the voltage Vp2 applied from the variable power supply 46 to the second measurement pump cell 41 in accordance with a difference between an actual value and the target value of the electromotive force V2 so that the electromotive force V2 is maintained at the target value. It is also suggested from the graph of FIG. 4 that the target value of the electromotive force V2 be preferably set to the value in the range of 1000 mV to 1500 mV.

The second measurement pump cell 41 operates in this manner, so that the oxygen partial pressure in the third chamber 61 is maintained at a much lower value than the oxygen partial pressure in each of the first chamber 20 and the second chamber 40. It is approximately 10⁻²⁰ atm when an equation V2=1000 mV holds true, for example. The measurement gas thus no longer substantially contains NOx, H₂O, CO₂, and oxygen while containing H₂ and CO (and further N₂).

The measurement gas containing H₂ and CO while not substantially containing NOx, H₂O, CO₂, and oxygen is introduced into the fourth chamber 63.

In the fourth chamber 63, oxygen is pumped in by operation of the third measurement pump cell 66, and only H₂ contained in the introduced measurement gas is selectively oxidized.

Pumping-in of oxygen is performed in the way that the third measurement pump cell control part 122c of the controller 110 sets a target value (control voltage) of the electromotive force V3 in the fourth chamber sensor cell 83 to a value in a range of 250 mV to 450 mV (preferably 350 mV) and feedback-controls the voltage Vp3 applied from the variable power supply 68 to the third measurement pump cell 66 in accordance with a difference between an actual value and the target value of the electromotive force V3 so that the electromotive force V3 is maintained at the target value.

The third measurement pump cell 66 operates in this manner, so that an oxidation (a combustion) reaction 2H₂+O₂→2H₂O is facilitated, and H₂O in an amount correlating with the amount of H₂O introduced through the gas inlet 10 is generated again in the fourth chamber 63. In the present embodiment, H₂O in the correlating amount means that the amount of H₂O introduced through the gas inlet 10 and the amount of H₂O generated again by oxidation of H₂ generated by decomposition of H₂O are the same or are within a certain error range allowable in terms of measurement accuracy.

The target value of the electromotive force V3 is set to the value in the range of 250 mV to 450 mV, so that the oxygen partial pressure in the fourth chamber 63 is maintained at a value in a range in which almost all H₂ is oxidized but CO is not oxidized. It is approximately 10⁻⁷ atm when an equation V3=350 mV holds true, for example.

Providing the third measurement electrode 64 as the cermet electrode containing the Pt—Au alloy having an Au concentration of 1 wt % or more and 50 wt % or less as the metal component as described above also contributes to improvement in selective H₂ oxidation property.

In addition, any measures to devise a shape (a width and a thickness), placement (a density), and the like of the heater 72 may be taken to further suppress a rise in temperature of the third measurement electrode 64.

In the gas sensor 100 according to the present embodiment operating in the above-mentioned manner, the concentrations of H₂O and CO₂ in the measurement gas are identified based on the oxygen pump current Ip2 flowing through the second measurement pump cell 41 during pumping-out of oxygen including reduction of H₂O and CO₂ and the oxygen pump current Ip3 flowing through the third measurement pump cell 66 during pumping-in of oxygen for oxidation of H₂.

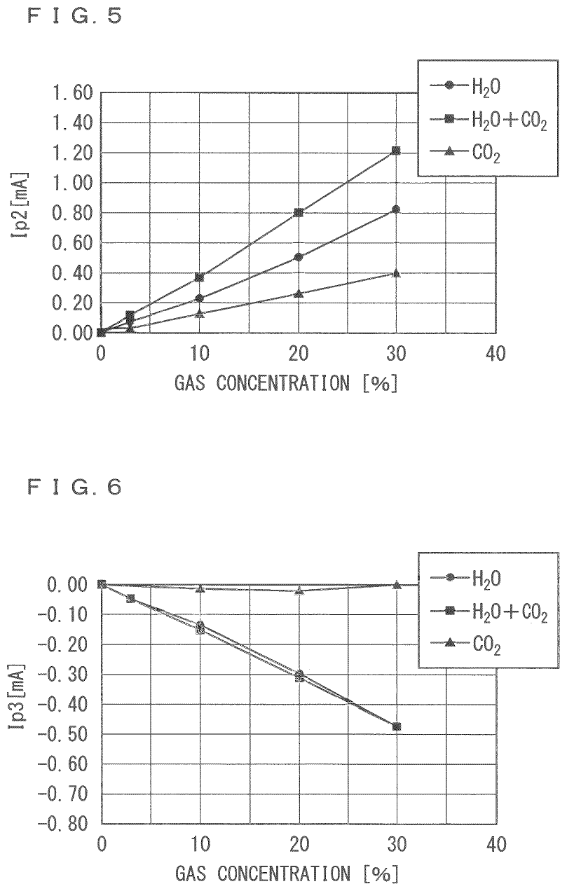

FIGS. 5 and 6 are diagrams showing, when only one of H₂O and CO₂ alone is contained in the measurement gas as a main sensing target gas component and when H₂O and CO₂ having equal concentrations are contained in the measurement gas as main sensing target gas components, dependence of the oxygen pump current Ip2 and the oxygen pump current Ip3 on a concentration of the sensing target gas component and the concentrations of the sensing target gas components.

In FIGS. 5 and 6, a graph when H₂O alone is contained as the sensing target gas component is shown by circles, a graph when CO₂ alone is contained as the sensing target gas component is shown by triangles, and a graph when H₂O and CO₂ having equal concentrations are contained as the sensing target gas components (described as “H₂O+CO₂” in the figure) is shown by squares. These graphs are obtained by operating the gas sensor 100 in an atmosphere of each of model gases containing a sensing target gas component having a known concentration and containing oxygen and nitrogen as the balance. The element driving temperature is 800° C. or more, and a temperature of each of the model gases is 200° C.

As can be seen from FIG. 5, the graph monotonically increases and is approximately linear when only H₂O is contained as the sensing target gas component and when only CO₂ is contained as the sensing target gas component.

Furthermore, a value of the oxygen pump current Ip2 when H₂O and CO₂ having equal concentrations are contained as the sensing target gas components is the sum of a value of the oxygen pump current Ip2 when H₂O alone is contained and a value of the oxygen pump current Ip2 when CO₂ alone is contained. It has also been confirmed that a value of the oxygen pump current Ip2 when H₂O and CO₂ have different proportions is the sum of a value of the oxygen pump current Ip2 when H₂O alone having a concentration in accordance with its proportion is contained and a value of the oxygen pump current Ip2 when CO₂ alone having a concentration in accordance with its proportion is contained, although it is not shown.

On the other hand, as shown in FIG. 6, a graph of the oxygen pump current Ip3 when only H₂O is contained as a sensing target gas component monotonically decreases (the absolute value thereof monotonically increases) and is approximately linear. The oxygen pump current Ip3 has a negative value because the oxygen pump current Ip3 flows in a direction in which oxygen is pumped in to oxidize H₂ generated by reduction in the third chamber 61 again, while the direction of the oxygen pump current when the third measurement pump cell 66 pumps out oxygen is assumed to be the positive direction of the oxygen pump current as described above.

In contrast, a value of the oxygen pump current Ip3 when only CO₂ is contained as a sensing target gas component is maintained approximately zero. This indicates that CO generated by reduction in the third chamber 61 is not oxidized again by operation of the third measurement pump cell 66.

A graph of the oxygen pump current Ip3 when H₂O and CO₂ having equal concentrations are contained as the sensing target gas components substantially coincides with the graph of the oxygen pump current Ip3 when H₂O alone is contained. This is consistent with the oxygen pump current Ip3 of approximately zero when only CO₂ is contained as the sensing target gas component. It has also been confirmed that a value of the oxygen pump current Ip3 when H₂O and CO₂ have different proportions substantially coincides with the graph of the oxygen pump current Ip3 when H₂O alone is contained and the graph of the oxygen pump current Ip3 when CO₂ alone is contained, although it is not shown. This means that the oxygen pump current Ip3 virtually depends only on the concentration of H₂O, and thus the concentration of H₂O can be identified once the oxygen pump current Ip3 is known.

In the present embodiment, the concentrations of H₂O and CO₂ in the measurement gas are measured using properties of the oxygen pump current Ip2 and the oxygen pump current Ip3 as described above. The oxygen pump current Ip2 and the oxygen pump current Ip3 during actual measurement using the gas sensor 100 are hereinafter also referred to as a total reducing current Ip2 and a water vapor equivalent current Ip3.

Specifically, prior to use of the gas sensor 100, characteristics data showing a relationship between the oxygen pump current Ip2 and a concentration of each gas when the measurement gas contains only one of H₂O and CO₂ and does not contain the other one of H₂O and CO₂ as shown in FIG. 5 (hereinafter also referred to as Ip2-H₂O data and Ip2-CO₂ data) and characteristics data showing a relationship between the oxygen pump current Ip3 and a concentration of H₂O when the measurement gas contains H₂O and does not contain CO₂ as shown in FIG. 6 (hereinafter also referred to as Ip3-H₂O data) are acquired in advance using model gases having known concentrations, and these pieces of characteristics data are stored in the controller 110. The Ip2-H₂O data and the Ip2-CO₂ data respectively have a value indicating a contribution of H₂O and a value indicating a contribution of CO₂ in the total reducing current Ip2.

The oxygen pump current Ip2 has a value in accordance with diffusion resistance provided to the measurement gas from the gas inlet 10 to the third chamber 61 of the sensor element 101, and the oxygen pump current Ip3 has a value in accordance with diffusion resistance provided to the measurement gas from the gas inlet 10 to the fourth chamber 63 of the sensor element 101. The Ip2-H₂O data, the Ip2-CO₂ data, and the Ip3-H₂O data thus strictly vary with each sensor element 101 of the gas sensor 100. These pieces of characteristics data are thus preferably identified for each gas sensor 100. As for gas sensors 100 manufactured under the same condition and from the same lot, however, characteristics data acquired for one particular gas sensor 100 may be applied to another gas sensor 100 from the same lot when it is confirmed that an error is within tolerance.

When actual measurement is performed using the gas sensor 100, the measurement gas is introduced into the sensor element 101 heated to the element driving temperature, and the adjustment pump cell 21, the first measurement pump cell 50, the second measurement pump cell 41, and the third measurement pump cell 66 operate in the above-mentioned manner. The water vapor concentration identification part 130H acquires the water vapor equivalent current Ip3 from the third measurement pump cell control part 122c and identifies a concentration of H₂O corresponding to the acquired value based on the Ip3-H₂O data.

Once the concentration of H₂O is identified, the carbon dioxide concentration identification part 130C acquires a value of the total reducing current Ip2 from the second measurement pump cell control part 122b and identifies a contribution of H₂O having the identified concentration in the total reducing current Ip2, that is, the amount of current due to reduction of H₂O in the total reducing current Ip2 based on the Ip2-H₂O data. The acquired value is subtracted from the value of the total reducing current Ip2 to identify a contribution of CO₂ in the total reducing current Ip2. A concentration of CO₂ corresponding to the contribution of CO₂ is finally identified based on the Ip2-CO₂ data.

In the gas sensor 100 according to the present embodiment, the concentrations of H₂O and CO₂ in the measurement gas are measured as described above.

Alternatively, a relationship between the water vapor equivalent current Ip3 and the oxygen pump current Ip2 corresponding to the contribution of H₂O in the total reducing current Ip2 may be identified in advance, characteristics data (hereinafter referred to as H₂O characteristics data) indicating the relationship may be stored in the controller 110, and the carbon dioxide concentration identification part 130C may identify the contribution of H₂O in the total reducing current Ip2 directly from the water vapor equivalent current Ip3 using the H₂O characteristics data.

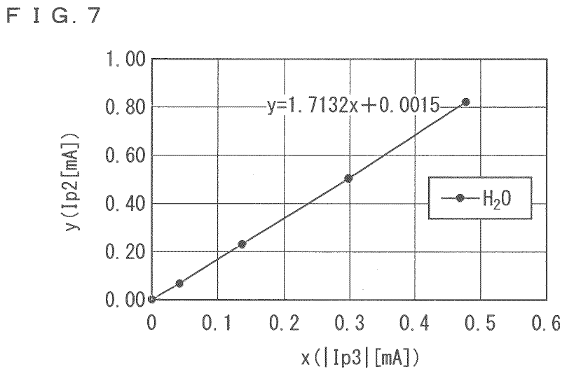

FIG. 7 is a diagram showing an example of the H₂O characteristics data. In FIG. 7, the absolute value of the water vapor equivalent current Ip3 is shown on an x-axis, and a value of the oxygen pump current Ip2 corresponding to the contribution of H₂O in the total reducing current Ip2 is shown on a y-axis. As shown in FIG. 7, there is a linear relationship between the water vapor equivalent current Ip3 and the contribution of H₂O in the total reducing current Ip2, so that a relation expressing the linear relationship is only required to be identified as the H₂O characteristics data.

Alternatively, a value of the y intercept of the relation should theoretically be zero and, in a case of the gas sensor 100 operating normally, is a value small enough to actually be considered zero. Thus, only the slope of the relation expressing the above-mentioned linear relationship may be stored in the controller 110 as the H₂O characteristics data, and the carbon dioxide concentration identification part 130C may use the product of a value of the slope and the water vapor equivalent current Ip3 as the contribution of H₂O in the total reducing current Ip2.

The slope in the H₂O characteristics data corresponds to a ratio of the diffusion resistance provided to the measurement gas from the gas inlet 10 to the fourth chamber 63 to the diffusion resistance provided to the measurement gas from the gas inlet 10 to the third chamber 61.

The concentration of oxygen is identified using the oxygen pump current Ip0 flowing through the adjustment pump cell 21 in parallel with identification of the concentrations of NOx, H₂O, and CO₂.

In the gas sensor 100 according to the present embodiment, oxygen is pumped out of the measurement gas introduced through the gas inlet 10 in the first chamber 20 by operation of the adjustment pump cell 21 as described above. Oxygen is pumped out so that NOx, H₂O, and CO₂ are not reduced, and the oxygen pump current Ip0 (hereinafter also referred to as an oxygen detection current Ip0) flowing in this case is substantially proportional to the concentration of oxygen contained in the measurement gas introduced through the gas inlet 10. That is to say, there is a linear relationship between the oxygen detection current Ip0 and the concentration of oxygen in the measurement gas. Data (Ip0-O₂ data) indicating the linear relationship is identified in advance using model gases having known oxygen concentrations and is stored in the controller 110.

During actual measurement using the gas sensor 100, the oxygen concentration identification part 130A acquires a value of the oxygen detection current Ip0 from the adjustment pump cell control part 121. A value of the concentration of oxygen corresponding to the acquired oxygen detection current Ip0 is identified with reference to the Ip0-O₂ data. The concentration of oxygen in the measurement gas is thereby identified.

As described above, in the gas sensor according to the present embodiment, the concentrations of H₂O and CO₂ can be measured when the measurement gas contains H₂O and CO₂ as in a conventional gas sensor. In addition, the concentration of NOx can simultaneously be measured. Furthermore, the concentration of oxygen can also accurately be obtained.

In addition, in a case of the gas sensor according to the present embodiment, H₂O and CO₂ are not reduced in the first chamber to be at a highest temperature during operation in contrast to the gas sensor in conventional technology, so that a voltage applied to the adjustment pump cell pumping out oxygen from the first chamber is suppressed to be lower than that in the gas sensor in conventional technology, thereby to suitably suppress cracking and blackening of the sensor element.

That is to say, according to the present embodiment, a multi-gas sensor having more long-term reliability than before and capable of simultaneously measuring many types of gases is implemented.

What is claimed is:

1. A gas sensor capable of measuring concentrations of a plurality of sensing target gas components, the gas sensor comprising:
a sensor element having a structure formed of an oxygen-ion conductive solid electrolyte; and
a controller controlling operation of the gas sensor, wherein
the sensor element comprises:
a gas inlet through which a measurement gas is introduced;
a first chamber, a second chamber, a third chamber, and a fourth chamber communicating sequentially from the gas inlet via different diffusion control parts;
an adjustment pump cell including an inner electrode formed to face the first chamber, an out-of-space pump electrode provided at a location other than a location in the first chamber, the second chamber, the third chamber, and the fourth chamber, and a portion of the solid electrolyte present between the inner electrode and the out-of-space pump electrode;
a first measurement pump cell including a first measurement electrode formed to face the second chamber, the out-of-space pump electrode, and a portion of the solid electrolyte present between the first measurement electrode and the out-of-space pump electrode;
a second measurement pump cell including a second measurement electrode formed to face the third chamber, the out-of-space pump electrode, and a portion of the solid electrolyte present between the second measurement electrode and the out-of-space pump electrode;
a third measurement pump cell including a third measurement electrode formed to face the fourth chamber, the out-of-space pump electrode, and a portion of the solid electrolyte present between the third measurement electrode and the out-of-space pump electrode; and
a heater heating the sensor element,
the inner electrode is a cermet electrode containing a Pt—Au alloy as a metal component, the Pt—Au alloy having an Au concentration of 0.5 wt % or more,
the first measurement electrode is another cermet electrode containing a Pt—Rh alloy as a metal component,
the adjustment pump cell pumps oxygen out of the measurement gas introduced through the gas inlet into the first chamber so that NOx, water vapor, and carbon dioxide contained in the measurement gas are not decomposed,
the first measurement pump cell pumps out oxygen from the second chamber so that substantially all NOx contained in the measurement gas introduced from the first chamber into the second chamber is reduced,
the second measurement pump cell pumps out oxygen from the third chamber so that substantially all water vapor and carbon dioxide contained in the measurement gas introduced from the second chamber into the third chamber are reduced,
the third measurement pump cell pumps oxygen into the fourth chamber to selectively oxidize, in the fourth chamber, hydrogen having been generated by reduction of water vapor and contained in the measurement gas introduced from the third chamber into the fourth chamber, and
the controller identifies:
a concentration of NOx contained in the measurement gas based on a magnitude of a NOx detection current as an oxygen pump current flowing between the first measurement electrode and the out-of-space pump electrode when oxygen is pumped out from the second chamber by the first measurement pump cell;
a concentration of water vapor contained in the measurement gas based on a value of a water vapor equivalent current as an oxygen pump current flowing between the third measurement electrode and the out-of-space pump electrode when hydrogen is oxidized with oxygen pumped into the forth chamber by the third measurement pump cell; and
a concentration of carbon dioxide contained in the measurement gas based on the value of the water vapor equivalent current and a value of a total reducing current as an oxygen pump current flowing between the second measurement electrode and the out-of-space pump electrode when water vapor and carbon dioxide are reduced by the second measurement pump cell pumping out oxygen from the third chamber.

2. The gas sensor according to claim 1, wherein
the controller stores
Ip1-NOx data indicating a relationship between the NOx detection current and a concentration of NOx, the Ip1-NOx data being identified in advance, and
identifies the concentration of NOx contained in the measurement gas based on the NOx detection current when NOx contained in the measurement gas is reduced and the Ip1-NOx data.

3. The gas sensor according to claim 2, wherein
the controller further stores:
Ip2-H₂O data indicating a relationship between an oxygen pump current flowing through the second measurement pump cell and a concentration of water vapor when the measurement gas contains water vapor and does not contain carbon dioxide, the Ip2-H₂O data being identified in advance;
Ip2-CO₂ data indicating a relationship between an oxygen pump current flowing through the second measurement pump cell and a concentration of carbon dioxide when the measurement gas contains carbon dioxide and does not contain water vapor, the Ip2-CO₂ data being identified in advance; and
Ip3-H₂O data indicating a relationship between an oxygen pump current flowing through the third measurement pump cell and a concentration of water vapor when the measurement gas contains water vapor and does not contain carbon dioxide, the Ip3-H₂O data being identified in advance,
and identifies:
a concentration of water vapor corresponding to the value of the water vapor equivalent current in the Ip3-H₂O data as the concentration of water vapor contained in the measurement gas, and
a contribution of water vapor by reduction in the total reducing current based on the identified concentration of water vapor contained in the measurement gas and the Ip2-H₂O data, and
a concentration of carbon dioxide corresponding to a difference value obtained by subtracting the contribution from the total reducing current in the Ip2-CO₂ data as the concentration of carbon dioxide contained in the measurement gas.

4. The gas sensor according to claim 2, wherein
the controller further stores:
Ip2-CO₂ data indicating a relationship between an oxygen pump current flowing through the second measurement pump cell and a concentration of carbon dioxide when the measurement gas contains carbon dioxide and does not contain water vapor, the Ip2-CO₂ data being identified in advance;
Ip3-H₂O data indicating a relationship between an oxygen pump current flowing through the third measurement pump cell and a concentration of water vapor when the measurement gas contains water vapor and does not contain carbon dioxide, the Ip3-H₂O data being identified in advance; and
H₂O characteristics data indicating a relationship between the water vapor equivalent current and an oxygen pump current corresponding to a contribution of water vapor in the total reducing current, the H₂O characteristics data being identified in advance,
and identifies:
a concentration of water vapor corresponding to the value of the water vapor equivalent current in the Ip3-H₂O data as the concentration of water vapor contained in the measurement gas,
a contribution of water vapor by reduction in the total reducing current based on the water vapor equivalent current and the H₂O characteristics data, and
a concentration of carbon dioxide corresponding to a difference value obtained by subtracting the contribution from the total reducing current in the Ip2-CO₂ data as the concentration of carbon dioxide contained in the measurement gas.

5. The gas sensor according to claim 1, wherein
the controller further identifies
a concentration of oxygen contained in the measurement gas based on a magnitude of a current flowing between the inner electrode and the out-of-space pump electrode when the adjustment pump cell pumps out oxygen from the first chamber.

6. The gas sensor according to of claim 1, wherein
the third measurement electrode is a cermet electrode containing a Pt—Au alloy as a metal component, and the Pt—Au alloy has an Au concentration of 1 wt % or more and 50 wt % or less.

7. A concentration measurement method of measuring concentrations of a plurality of sensing target gas components using a gas sensor, wherein
the gas sensor includes a sensor element having an elongated planar structure formed of an oxygen-ion conductive solid electrolyte,
the sensor element comprises:
a gas inlet through which a measurement gas is introduced;
a first chamber, a second chamber, a third chamber, and a fourth chamber communicating sequentially from the gas inlet via different diffusion control parts;
an adjustment pump cell including an inner electrode formed to face the first chamber, an out-of-space pump electrode provided at a location other than a location in the first chamber, the second chamber, the third chamber, and the fourth chamber, and a portion of the solid electrolyte present between the inner electrode and the out-of-space pump electrode;
a first measurement pump cell including a first measurement electrode formed to face the second chamber, the out-of-space pump electrode, and a portion of the solid electrolyte present between the first measurement electrode and the out-of-space pump electrode;
a second measurement pump cell including a second measurement electrode formed to face the third chamber, the out-of-space pump electrode, and a portion of the solid electrolyte present between the second measurement electrode and the out-of-space pump electrode;
a third measurement pump cell including a third measurement electrode formed to face the fourth chamber, the out-of-space pump electrode, and a portion of the solid electrolyte present between the third measurement electrode and the out-of-space pump electrode; and
a heater heating the sensor element,
the inner electrode is a cermet electrode containing a Pt—Au alloy as a metal component, the Pt—Au alloy having an Au concentration of 0.5 wt % or more,
the first measurement electrode is another cermet electrode containing a Pt—Rh alloy as a metal component, and
the concentration measurement method using the gas sensor comprises:
a) pumping, using the adjustment pump cell, oxygen out of the measurement gas introduced through the gas inlet into the first chamber so that NOx, water vapor, and carbon dioxide contained in the measurement gas are not decomposed;
b) pumping out, using the first measurement pump cell, oxygen from the second chamber so that substantially all NOx contained in the measurement gas introduced from the first chamber into the second chamber is reduced;
c) pumping out, using the second measurement pump cell, oxygen from the third chamber so that substantially all water vapor and carbon dioxide contained in the measurement gas introduced from the second chamber into the third chamber are reduced;
d) pumping, using the third measurement pump cell, oxygen into the fourth chamber to selectively oxidize, in the fourth chamber, hydrogen having been generated by reduction of water vapor and contained in the measurement gas introduced from the third chamber into the fourth chamber;
e) identifying a concentration of NOx contained in the measurement gas based on a magnitude of a NOx detection current as an oxygen pump current flowing between the first measurement electrode and the out-of-space pump electrode when NOx is reduced by pumping out oxygen from the second chamber using the first measurement pump cell;
f) identifying a concentration of water vapor contained in the measurement gas based on a value of a water vapor equivalent current as an oxygen pump current flowing between the third measurement electrode and the out-of-space pump electrode when hydrogen is oxidized with oxygen pumped into the fourth chamber using the third measurement pump cell; and
g) identifying a concentration of carbon dioxide contained in the measurement gas based on the value of the water vapor equivalent current and a value of a total reducing current as an oxygen pump current flowing between the second measurement electrode and the out-of-space pump electrode when water vapor and carbon dioxide are reduced by pumping out oxygen from the third chamber using the second measurement pump cell.

8. The concentration measurement method using the gas sensor according to claim 7, further comprising
h) prior to the steps a) to g), identifying Ip1-NOx data in advance, the Ip1-NOx data indicating a relationship between the NOx detection current and a concentration of NOx, wherein
in the step e), the concentration of NOx contained in the measurement gas is identified based on the NOx detection current when NOx contained in the measurement gas is reduced and the Ip1-NOx data.

9. The concentration measurement method using the gas sensor according to claim 8, wherein
the step h) further comprises identifying:
Ip2-H₂O data indicating a relationship between an oxygen pump current flowing through the second measurement pump cell and a concentration of water vapor when the measurement gas contains water vapor and does not contain carbon dioxide;
Ip2-CO₂ data indicating a relationship between an oxygen pump current flowing through the second measurement pump cell and a concentration of carbon dioxide when the measurement gas contains carbon dioxide and does not contain water vapor; and
Ip3-H₂O data indicating a relationship between an oxygen pump current flowing through the third measurement pump cell and a concentration of water vapor when the measurement gas contains water vapor and does not contain carbon dioxide,
in the step f), a concentration of water vapor corresponding to the value of the water vapor equivalent current in the Ip3-H₂O data is identified as the concentration of water vapor contained in the measurement gas, and
in the step g), a contribution of water vapor by reduction in the total reducing current is identified based on the concentration of water vapor contained in the measurement gas identified in the step f) and the Ip2-H₂O data, and a concentration of carbon dioxide corresponding to a difference value obtained by subtracting the contribution from the total reducing current in the Ip2-CO₂ data is identified as the concentration of carbon dioxide contained in the measurement gas.

10. The concentration measurement method using the gas sensor according to claim 8, wherein
the step h) further comprises identifying:
Ip2-CO₂ data indicating a relationship between an oxygen pump current flowing through the second measurement pump cell and a concentration of carbon dioxide when the measurement gas contains carbon dioxide and does not contain water vapor;
Ip3-H₂O data indicating a relationship between an oxygen pump current flowing through the third measurement pump cell and a concentration of water vapor when the measurement gas contains water vapor and does not contain carbon dioxide; and
H₂O characteristics data indicating a relationship between the water vapor equivalent current and an oxygen pump current corresponding to a contribution of water vapor in the total reducing current,
in the step f), a concentration of water vapor corresponding to the value of the water vapor equivalent current in the Ip3-H₂O data is identified as the concentration of water vapor contained in the measurement gas, and
in the step g), a contribution of water vapor by reduction in the total reducing current is identified based on the water vapor equivalent current and the H₂O characteristics data, and a concentration of carbon dioxide corresponding to a difference value obtained by subtracting the contribution from the total reducing current in the Ip2-CO₂ data is identified as the concentration of carbon dioxide contained in the measurement gas.

11. The concentration measurement method using the gas sensor according to claim 7, further comprising
i) identifying a concentration of oxygen contained in the measurement gas based on a magnitude of a current flowing between the inner electrode and the out-of-space pump electrode when oxygen is pumped out from the first chamber using the adjustment pump cell.

12. The concentration measurement method using the gas sensor according to claim 7, wherein
the third measurement electrode is a cermet electrode containing a Pt—Au alloy as a metal component, and the Pt—Au alloy has an Au concentration of 1 wt % or more and 50 wt % or less.

13. The gas sensor according to claim 2, wherein
the controller further identifies
a concentration of oxygen contained in the measurement gas based on a magnitude of a current flowing between the inner electrode and the out-of-space pump electrode when the adjustment pump cell pumps out oxygen from the first chamber.

14. The gas sensor according to claim 3, wherein
the controller further identifies
a concentration of oxygen contained in the measurement gas based on a magnitude of a current flowing between the inner electrode and the out-of-space pump electrode when the adjustment pump cell pumps out oxygen from the first chamber.

15. The gas sensor according to claim 4, wherein
the controller further identifies
a concentration of oxygen contained in the measurement gas based on a magnitude of a current flowing between the inner electrode and the out-of-space pump electrode when the adjustment pump cell pumps out oxygen from the first chamber.

16. The gas sensor according to of claim 2, wherein
the third measurement electrode is a cermet electrode containing a Pt—Au alloy as a metal component, and the Pt—Au alloy has an Au concentration of 1 wt % or more and 50 wt % or less.

17. The gas sensor according to of claim 3, wherein
the third measurement electrode is a cermet electrode containing a Pt—Au alloy as a metal component, and the Pt—Au alloy has an Au concentration of 1 wt % or more and 50 wt % or less.

18. The gas sensor according to of claim 4, wherein
the third measurement electrode is a cermet electrode containing a Pt—Au alloy as a metal component, and the Pt—Au alloy has an Au concentration of 1 wt % or more and 50 wt % or less.

19. The concentration measurement method using the gas sensor according to claim 8, further comprising
i) identifying a concentration of oxygen contained in the measurement gas based on a magnitude of a current flowing between the inner electrode and the out-of-space pump electrode when oxygen is pumped out from the first chamber using the adjustment pump cell.

20. The concentration measurement method using the gas sensor according to claim 9, further comprising
i) identifying a concentration of oxygen contained in the measurement gas based on a magnitude of a current flowing between the inner electrode and the out-of-space pump electrode when oxygen is pumped out from the first chamber using the adjustment pump cell.
